(19) United States
(12) Patent Application Publication
(10) Pub. No.: US 2019/0365862 A1
(43) Pub. Date: Dec. 5, 2019

(54) COMBINATION THERAPIES FOR ERADICATING FLAVIVIRUS INFECTIONS IN SUBJECTS

(71) Applicant: Temple University - of the Commonwealth System of Higher Education, Philadelphia, PA (US)

(72) Inventors: Kamel Khalili, Bala Cynwyd, PA (US); Ilker K. Sariyer, Broomall, PA (US); Hassen Wollebo, Philadelphia, PA (US)

(21) Appl. No.: 16/341,481

(22) PCT Filed: Oct. 12, 2017
(86) PCT No.: PCT/US17/56267

§ 371 Date: Apr. 12, 2019

Related U.S. Application Data
(60) Provisional application No. 62/406,976, filed on Oct. 12, 2016.

(51) Int. Cl. A61K 38/21; A61K 31/7105; A61K 38/20; A61K 38/46; C12N 15/86; A61K 48/00

(52) U.S. Cl. CPC A61K 38/217; A61K 31/7105; A61K 48/0016; A61K 38/465; C12N 15/86; A61K 38/20

(57) ABSTRACT

Compositions that specifically cleave target sequences in Flavivirus, for example Zika virus include a Clustered Regularly Interspaced Short Palindromic Repeat (CRISPR) associated endonuclease, a guide RNA sequence complementary to a target sequence in a Zika virus and an anti-viral agent. These compositions are administered to a subject for treating an infection or at risk for contracting a Zika virus infection.

### RELATED APPLICATION

This application claims priority under 35 U.S.C. § 119 to U.S. Patent Application No. 62/406,976 filed Oct. 12, 2016, the entire contents of which is hereby expressly incorporated by reference herein.

### FIELD OF THE INVENTION

The present invention relates to compositions that specifically cleave target sequences in Flavivirus, for example, Zika virus. Such compositions, which include nucleic acids encoding a Clustered Regularly Interspaced Short Palindromic Repeat (CRISPR) associated endonuclease, a guide RNA sequence complementary to a target sequence in a Zika virus and an anti-viral agent, can be administered to a subject having or at risk for contracting a Zika virus infection.

### BACKGROUND

Once a rare virus found in the rhesus monkey in the Zika forest in Uganda, the Zika virus has become an urgent public health concern in many countries and has been associated with microcephaly in neonates and Guillain-Barré syndrome in adults (Dick et al. 1952. Trans R Soc Trop Med Hyg 46: 509-520; Broutet et al, 2016, N Engl J Med (In Press); Chan et al, 2016, J Infect (In Press); Lazear H. M. and Diamond M. S., 2016, J Virol April 29; 90(10):4864-75); Vogel, 2016 Science 351: 1123-1124). The virus remained obscure with few human cases confined to Africa and Asia (Moore et al, 1975, Ann Trop Med Parasitol 69: 49-64) until the Asian strain caused Zika outbreaks in Micronesia in 2007 (Haddow et al, 2012, Bull World Health Organ 31: 57-69) & French Polynesia in 2013-2014 (Cao-Lormeau et al, 2014, Emerg Infect Dis 20: 1085-1086).

In French Polynesia (2013-2014), the outbreak spread to other Pacific Islands: New Caledonia, Cook Islands, Easter Island, Vanuatu, and Solomon Islands (Musso D. 2015, Emerg Infect Dis 21: 1887). Zika virus then spread to Brazil by an unknown means of transmission but phylogenetic studies showed that closest strain to the one that emerged in Brazil was from samples from French Polynesia and spread in the Pacific Islands (Campos et al, 2015, Emerg Infect Dis 21: 1885-1886; Musso, D. 2015, Emerg Infect Dis 21: 1887). The first report of autochthonous Zika transmission in the Americas was in March 2015 in Rio Grande do Norte, Northeast Brazil (Zanluca et al, 2015; Hennessey et al, 2016). The epidemic has spread in Brazil with now ˜1,300,000 suspected cases in late 2015 (Hennessey et al, 2016, MMWR Morb Mortal Wkly Rep 65: 55-58; Bogoch et al, 2016, Lancet 387: 335-336). Already Zika has begun to spread beyond Brazil and further spread of is anticipated with imported cases already been reported in the US, Europe and other countries where travelers are returning after visiting Latin America and the Caribbean (Hennessey et al, 2016, MMWR Morb Mortal Wkly Rep 65: 55-58; Hills et al, 2016, MMWR Morb Mortal Wkly Rep 65: 215-216).

The rapid advance of the virus and the reported high rates of microcephaly and Guillain-Barré syndrome associated with Zika infection in Polynesia and Brazil have raised concerns that it represents an evolving neuropathic and teratogenic public health threat. The Pan American Health Organization predicts that Zika virus will spread to eventually reach all areas where Aedes mosquitoes are endemic (Malone et al, 2016, PLoS Negl Trop Dis 10: e0004530). There are no licensed vaccines, therapeutic or preventive drugs available for Zika virus and hence the development and deployment of countermeasures are urgently needed.

Ominously, it now appears that the virus may be able to be transmitted by means other than the Aedes mosquito (Lazear and Diamond, 2016, J Virol JVI.00252-16 (In Press)). Firstly, since Zika is a blood borne pathogen, it is possible that a Zika-infected blood donor could contaminate the blood supply and cases of Zika transmission through transfusion have been reported in Brazil (Lazear and Diamond, 2016). The efficiency of the transmission of Zika virus by transfusions is still unknown and additional studies are needed (Musso et al, 2014, Euro Surveill 19(14) pii: 20761; Marano et al, 2016, Blood Transfus 14: 95-100). Screening of donated blood by PCR-based tests as is done for West Nile Virus would prevent this possibility if these become available or, if not, application of strategies for inactivation of the virus (Kleinman, S 2015, Curr Opin Hematol 22: 547-553; Aubry et al, 2016, Transfusion 56: 33-40). Secondly, Zika can be transmitted sexually (Foy et al, 2011, Emerg Infect Dis 17: 880-882; Musso et al, 2015, Emerg Infect Dis 21: 1887; Hills et al, 2016, MMWR Morb Mortal Wkly Rep 65: 215-216) and in these cases, virus was transmitted from infected men to their female partners. Accordingly, Zika viral RNA can be detected in semen (Musso et al, 2015, Emerg Infect Dis 21: 1887; Mansuy et al, 2016, Lancet Infect Dis (In Press)) and in one report, the RNA virus load was about 100,000 times that of matched blood or urine samples at a time of more than 2 weeks after the onset of symptoms. Lastly, perinatal transmission of Zika has been reported but it is not known if this occurred in utero, via breast milk or by a blood borne route (Besnard et al, 2014, Euro Surveill 19(13) pii: 20751). This may be particularly important given the association of Zika with neonatal abnormalities such as microcephaly.

### SUMMARY

Embodiments of the invention are directed to compositions for eradicating a Flavivirus, in vitro or in vivo. Methods of treatment or prevention of an infection comprises the use of the compositions.

In certain embodiments, a composition for eradicating a flavivirus in vitro or in vivo, the composition comprises an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Flavivirus genome; an antiviral agent, or combinations thereof. The Flavivirus comprises: dengue virus, tick-borne encephalitis virus, West Nile virus, yellow fever virus, Japanese encephalitis virus, Kyasanur Forest disease virus, Alkhurma hemorrhagic fever virus, Omsk hemorrhagic fever virus, or Zika virus.

In certain embodiments, the Flavivirus is Zika virus.

In certain embodiments, the antiviral agent comprises: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, ribozymes, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

In certain embodiments, the antiviral agent comprises interferon-alpha (IFNα), interferon-beta (IFNβ), interferon-gamma (IFNγ), interferon tau (IFNτ), interferon omega (IFNω), or combinations thereof. In some embodiments, the anti-viral agent is interferon-gamma (IFNγ).

In certain embodiments, the target nucleic acid sequence comprises one or more nucleic acid sequences in coding and non-coding nucleic acid sequences of the Flavivirus genome. In embodiments, the target nucleic acid sequence comprises one or more sequences within a sequence encoding structural proteins, non-structural proteins or combinations thereof. The sequences encoding structural proteins comprise nucleic acid sequences encoding a capsid protein (C), precursor viral membrane protein (prM), viral membrane protein (M), envelop protein (E) or combinations thereof.

The sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In certain embodiments, the gRNA sequence has at least a 75% sequence identity to one or more sequences complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or any combination thereof.

In certain embodiments, the gRNA has at least a 75% sequence identity to any one or more of SEQ ID NOS: 1-27. In other embodiments, a gRNA comprises any one or more of SEQ ID NOS: 1-27. In certain embodiments, the composition further comprises a short proto-spacer adjacent motif (PAM)-presenting DNA oligonucleotide sequence (PAMmer) wherein the PAMmer comprises a PAM and additional Flavivirus nucleic acid sequences downstream of target Flavivirus nucleic acid sequences of the gRNA.

In certain embodiments, the guide RNA sequences are in single or multiplex configurations. The guide RNA sequences are encoded by the same vector encoding the CRISPR/Cas molecule or are encoded by separate vectors. In certain embodiments, a gRNA comprises one or more modified nucleic acid bases or chimeric sequences.

In certain embodiments, the composition further comprises an anti-pyretic agent, anti-inflammatory agent, chemotherapeutic agent, or combinations thereof.

In other embodiments, a method of eradicating a Flavivirus genome in a cell or a subject, comprises contacting the cell or administering to the subject, a therapeutically effective amount of a pharmaceutical composition comprising: an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Flavivirus genome; an antiviral agent, or combinations thereof.

In certain embodiments, a method of inhibiting replication of a Flavivirus in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Flavivirus genome; an antiviral agent, an anti-pyretic agent, anti-inflammatory agent, chemotherapeutic agent, or combinations thereof. The antiviral agent comprises: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

In certain embodiments, a composition for eradicating a flavivirus in vitro or in vivo, the composition comprising: a gene editing agent; at least one guide nucleic acid sequence (gNAS), the gNAS being complementary to a target nucleic acid sequence in a Flavivirus genome; an antiviral agent, or combinations thereof.

In certain embodiments, the gene-editing agent comprises: Argonaute family of endonucleases, clustered regularly interspaced short palindromic repeat (CRISPR) nucleases, zinc-finger nucleases (ZFNs), transcription activator-like effector nucleases (TALENs), meganucleases, endo- or exo-nucleases, or combinations thereof.

In certain embodiments, the gNAS comprises a ribonucleic acid (RNA) or deoxyribonucleic acid (DNA). In some embodiments, the gNAS comprises one or more modified nucleic acid bases or chimeric regions. In certain embodiments, the gene editing agent and the at least one gNAS is encoded by the same vector or separate vectors. In certain embodiments, the guide NAS sequences are in single or multiplex configurations.

In certain embodiments, a method of treating a subject infected with a Zika virus, comprises administering to the subject, a pharmaceutical composition comprising a therapeutically effective amount of an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Zika virus genome; and, an antiviral agent. In certain embodiments, the antiviral agent comprises interferon-alpha (IFNα), interferon-beta (IFNβ), interferon-gamma (IFNγ), interferon tau (IFNτ), interferon omega (IFNω), analogs or combinations thereof.

In other embodiments, a pharmaceutical composition comprises a therapeutically effective amount of an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Zika virus genome; and, an antiviral agent. The antiviral agent comprises interferon-alpha (IFNα), interferon-beta (IFNβ), interferon-gamma (IFNγ), interferon tau (IFNτ), interferon omega (IFNω), analogs or combinations thereof. In certain embodiments, a gRNA comprises one or more modified nucleic acid bases or chimeric sequences. In certain embodiments, the guide RNA sequences are in single or multiplex configurations. In certain embodiments, the target nucleic acid sequence comprises one or more nucleic acid sequences in coding and non-coding nucleic acid sequences of the Zika virus genome. The target nucleic acid sequence comprises one or more sequences within a sequence encoding structural proteins, non-structural proteins or combinations thereof. In certain embodiments, sequences encoding structural proteins comprise nucleic acid sequences encoding a capsid protein (C), precursor viral membrane protein (prM), viral membrane protein (M), envelop protein (E) or combinations thereof. The sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof. In certain embodiments, the at least one gRNA sequence has at least a 75% sequence identity to at least one sequence, the sequence being complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof. In certain embodiments, a gRNA has at least a 75% sequence identity to any one or more of SEQ ID NOS: 1-27. In certain embodiments, gRNA comprises any one or more of SEQ ID NOS: 1-27.

In certain embodiments, the pharmaceutical composition further comprises an anti-pyretic agent, anti-inflammatory agent, chemotherapeutic agent, or combinations thereof.

Other aspects are described infra.

### BRIEF DESCRIPTION OF THE DRAWINGS

FIG. 1 is a graph showing that Zika virus replication and viral propagation is suppressed by the combination of IFN-gamma and CRISPR/Cas9 mediated gene editing strategy.

### DETAILED DESCRIPTION

Embodiments of the invention are directed to compositions for eradicating a flavivirus, in vitro or in vivo. The compositions comprise a gene editing agent, a guide nucleic acid sequence for specific targeting of the gene editing agent, at least one anti-viral agent. In particular, the compositions comprise isolated nucleic acid sequences encoding a Clustered Regularly Interspaced Short Palindromic Repeat (CRISPR)-associated endonuclease, at least one guide RNA (gRNA), the gRNA being complementary to a target nucleic acid sequence in a Flavivirus genome and an anti-viral agent.

The isolated nucleic acid can be encoded by a vector or encompassed in one or more delivery vehicles and formulations as described in detail below.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, ribozymes, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

The term “antibody” as used herein comprises one or more virus specific binding domains which bind to and aid in the immune mediated-destruction and clearance of the virus, e.g. Zika virus. The antibody or fragments thereof, comprise IgA, IgM, IgG, IgE, IgD or combinations thereof.

The term “eradication” of the Flavivirus, e.g. Zika virus, as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “immunoregulatory” or “immune cell modulator” is meant a compound, composition or substance that is immunogenic (i.e. stimulates or increases an immune response) or immunosuppressive (i.e. reduces or suppresses an immune response). “Cells of the immune system” or “immune cells”, is meant to include any cells of the immune system that may be assayed or involved in mounting an immune response, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types. The functions or responses to an antigen can be measured by any type of assay, e.g. RIA, ELISA, FACS, Western blotting, etc.

The term “induces or enhances an immune response” is meant causing a statistically measurable induction or increase in an immune response over a control sample to which the peptide, polypeptide or protein has not been administered. Conversely, “suppression” of an immune response is a measurable decrease in an immune response over a control sample to which the peptide, polypeptide or protein has been administered, for example, as in the case of suppression of an immune response in an auto-immune scenario. Preferably the induction or enhancement of the immune response results in a prophylactic or therapeutic response in a subject. Examples of immune responses are increased production of type I IFN, increased resistance to viral and other types of infection by alternate pathogens. The enhancement of immune responses to viruses (anti-virus responses), or the development of vaccines to prevent virus infections or eliminate existing viruses.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “percent sequence identity” or having “a sequence identity” refers to the degree of identity between any given query sequence and a subject sequence.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Compositions for Eradication of Flavivirus in Cells or Subjects

Zika virus is an emerging virus with important public health consequences. Zika virus disease is caused by the Zika virus, which is spread to people primarily through the bite of an infected mosquito (Aedes aegypti and Aedes albopictus). Zika virus is an arbovirus (arthropod-borne virus) and a member of the family Flaviviridae, genus Flavivirus. Zika virus is related to other human flaviviruses that cause significant pathology including yellow fever, dengue, tick-borne encephalitis, Saint Louis encephalitis, Japanese encephalitis and West Nile viruses and is most closely related to Spondweni virus (Faye et al, 2014, PLoS Negl Trop Dis 8(1): e2636). Zika virions are enveloped and icosahedral, and contain a nonsegmented, single-stranded, positive-sense RNA genome, which is about 11 Kb in length and expresses seven nonstructural proteins and three structural proteins that are encoded as a single polyprotein in a unique long open reading frame containing all of the structural protein genes at the 5′ portion of the genome and the nonstructural (NS) protein genes at the 3′ portion. The genome organization of flaviviruses, concerning the protein expression order is:

5′-C-prM-E-NS1-NS2a-NS2b-NS3-NS4a-NS4b-NS5-3′

The capsid protein (C) is 13 kDa in size, highly basic and complexes with the viral RNA in the nucleocapsid while the outer membrane of the virion is a lipid bilayer containing the viral membrane protein (M) and envelope protein (E). The M protein is expressed as a larger glycosylated precursor protein (prM) while the E protein may or may not be glycosylated and this is a determinant of neuroinvasion, acting to increase both axonal and trans-epithelial transportation (Neal, 2014, J Infect 69: 203-215). The genomic RNA of flaviviruses lacks a poly-A tail at the 3′ end (Wengler and Wengler, 1981, Virology 13: 544-555) and has an m⁷gpppAmpN2 at the 5′ end (Cleaves and Dubin, 1979, Virology 96: 159-165). Several regions within the genome of flaviviruses have a highly conserved structure including a 90-120 nucleotide stretch near the 3′ end, which is thought to form a stable hairpin loop (Brinton et al, 1986, Virology 153: 113-121). Mutational analysis of this region in Dengue virus revealed that it has an essential role in viral replication (Zeng et al, 1998, J Virol 72: 7510-7522).

Flavivirus particles bind to the surface of target cells by interactions between viral surface glycoproteins and cellular cell surface receptors. Virions undergo receptor-mediated endocytosis and are internalized into clathrin-coated pits (Gollins and Porterfield, 1985, J Gen Virol 66: 1969-1982). Uncoating of the virus envelope releases the viral RNA into the cytoplasm and also activates the host cell innate response followed by complex interplay between virus and host where virus co-opts the host cytoplasmic membranes for replication of its genome and the host attempts to control infection with several responses including interferon release, the unfolded protein/endoplasmic reticulum response, autophagy and apoptosis (Nain et al, 2016, Rev Med Virol 26: 129-141). Translation of viral proteins from the viral RNA occurs from the long open reading frame to produce a large polyprotein that is cleaved co- and posttranslationally into the individual viral proteins and leads to replication of the viral genome.

The viral RNA, structural and non-structural proteins and some host proteins are involved in the assembly of the viral replication complex in vesicle packages in the cytoplasm of infected cells (Lindenbach and Rice, 2003, Adv Virus Res 59: 23-61). Replication initiates with the synthesis of a negative-strand RNA, which then serves as a template for the synthesis of copies of the positive-strand genomic RNA in an asymmetric fashion such that there is 10- to 100-fold excess of positive strands over negative strands (Cleaves et al, 1981, Virology 111: 73-83). Replication requires the activities of several of the viral nonstructural (NS) proteins. NS3 consists of an N-terminal serine protease and a C-terminal helicase with NS3 protease activity requiring NS2B as a cofactor, and cleaving the viral polyprotein at several positions between the NS proteins. The NS3 helicase domain has helicase, RNA-stimulated nucleoside triphosphate hydrolase and 5′-RNA triphosphatase activities with the helicase activity required for unwinding the double-stranded RNA intermediate formed during genome synthesis and the 5′-RNA triphosphatase activity required for 5′-RNA cap formation. NS5 contains a C-terminal RNA-dependent RNA polymerase (RdRp) activity that is involved in viral genome replication and carries out both (−) and (+) strand RNA synthesis (Klema et al, 2015, Viruses 7: 4640-4656). Virus particles assemble by budding into the endoplasmic reticulum and nascent virus particles traverse the host secretory pathway, where virion maturation occurs followed by release from the cell (Lindenbach and Rice, 2003, Adv Virus Res 59: 23-61). Zika virus can be cultured in suckling mice and also grows well in Vero cells (Way et al, 1976, J Gen Virol 30: 123-130). In infections in vivo, flaviviruses can target a variety of cell types including dendritic cells, macrophages, endothelial cells and neuronal cells (Hidari and Suzuki, 2011, Trop Med Health 39(4 Suppl): 37-43; Dalrymple and Mackow, 2014, Curr Opin Virol 7:134-140; Neal, 2014, J Infect 69: 203-215).

No clinically approved therapy is currently available for the treatment of Zika or indeed any other flavivirus infection (Lim et al., 2013, Antiviral Res 100: 500-519). Over the past decade, significant effort has been made towards dengue drug discovery. Due to the similarity between Zika virus and dengue virus, it is possible that knowledge from dengue drug discovery could be applied to Zika virus. Several approaches are possible, e.g., high-throughput screening using virus replication assays or viral enzyme assays, structure-based in silico docking and rational design strategies and repurposing hepatitis C virus inhibitors for Zika. The development of antivirals should focus on distinctive features of Zika molecular biology that can be exploited. For example, Zika NS3 protein has a protease activity that is necessary for the viral life cycle and this may be a viable target for small molecule antiviral inhibitors. In this regard, the inhibitors of the NS3/4A protease of Hepatitis C, telaprevir and boceprevir, revolutionize the management of hepatitis C genotype 1 patients (Vermehren and Sarrazin, 2011, Eur J Med Res 16: 303-314). NS3 also has a 5′-RNA triphosphatase activity required for 5′-RNA cap formation and NS5 contains a C-terminal RNA-dependent RNA polymerase (RdRp) activity as described above and these are also potential targets for the development of small molecule antiviral inhibitors (Lim et al, 2015, Antiviral Res 100: 500-519; Luo et al, 2015, Antiviral Res 118: 148-158). Finally, the advent of methodologies such as the CRISPR/Cas9 system that are specifically able to target nucleotide sequences within viral genomes has provided an effective, specific, and versatile weapon against human DNA viruses (White et al, 2015, Discov Med 19: 255-262).

Accordingly, the compositions disclosed herein, include nucleic acids encoding a gene editing agent, for example, CRISPR-associated endonuclease, such as Cas9. In some embodiments, one or more guide RNAs that are complementary to a target sequence of a Flavivirus may also be encoded.

Methods of the invention may be used to remove viral or other foreign genetic material from a host organism, without interfering with the integrity of the host's genetic material. A nuclease may be used to target viral nucleic acid, thereby interfering with viral replication or transcription or even excising the viral genetic material from the host genome. The nuclease may be specifically targeted to remove only the viral nucleic acid without acting on host material either when the viral nucleic acid exists as a particle within the cell or when it is integrated into the host genome. Targeting the viral nucleic acid can be done using a sequence-specific moiety such as a guide RNA that targets viral genomic material for destruction by the nuclease and does not target the host cell genome. In some embodiments, a CRISPR/Cas nuclease and guide RNA (gRNA) that together target and selectively edit or destroy viral genomic material is used. The CRISPR (clustered regularly interspaced short palindromic repeats) is a naturally-occurring element of the bacterial immune system that protects bacteria from phage infection. The guide RNA localizes the CRISPR/Cas complex to a viral target sequence. Binding of the complex localizes the Cas endonuclease to the viral genomic target sequence causing breaks in the viral genome. Other nuclease systems can be used including, for example, zinc finger nucleases, transcription activator-like effector nucleases (TALENs), meganucleases, or any other system that can be used to degrade or interfere with viral nucleic acid without interfering with the regular function of the host's genetic material.

The compositions may be used to target viral nucleic acid in any form or at any stage in the viral life cycle. The targeted viral nucleic acid may be present in the host cell as independent particles. In a preferred embodiment, the viral infection is latent and the viral nucleic acid is integrated into the host genome. Any suitable viral nucleic acid may be targeted for cleavage and digestion.

Gene Editing Agents:

Compositions of the invention include at least one gene editing agent, comprising CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

The composition can also include C2c2—the first naturally-occurring CRISPR system that targets only RNA. The Class 2 type VI-A CRISPR-Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. These results demonstrate the capability of C2c2 as a new RNA-targeting tools.

C2c2 can be programmed to cleave particular RNA sequences in bacterial cells. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner- and manipulate gene function more broadly.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 could have multiple applications, including treatment of genetic illnesses and degenerative conditions. As referenced above, Argonaute is another potential gene editing system.

Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K. S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss)RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

Accordingly, in certain embodiments, Argonaute endonucleases comprise those which associate with single stranded RNA (ssRNA) or single stranded DNA (ssDNA). In certain embodiments, the Argonaute is derived from Natronobacterium gregoryi. In other embodiments. the Natronobacterium gregoryi Argonaute (NgAgo) is a wild type NgAgo, a modified NgAgo, or a fragment of a wild type or modified NgAgo. The NgAgo can be modified to increase nucleic acid binding affinity and/or specificity, alter an enzymatic activity, and/or change another property of the protein. For example, nuclease (e.g., DNase) domains of the NgAgo can be modified, deleted, or inactivated.

The wild type NgAgo sequence can be modified. The NgAgo nucleotide sequence can be modified to encode biologically active variants of NgAgo, and these variants can have or can include, for example, an amino acid sequence that differs from a wild type NgAgo by virtue of containing one or more mutations (e.g., an addition, deletion, or substitution mutation or a combination of such mutations). One or more of the substitution mutations can be a substitution (e.g., a conservative amino acid substitution). For example, a biologically active variant of an NgAgo polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type NgAgo polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the NgAgo amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Another gene editing agent is human WRN, a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease. RNase P RNA are another potential endonuclease for use with the present invention.

CRISPR-Associated Endonucleases:

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mechanism through which CRISPR/Cas9-induced mutations inactivate the provirus can vary. For example, the mutation can affect proviral replication, and viral gene expression. The mutation can comprise one or more deletions. The size of the deletion can vary from a single nucleotide base pair to about 10,000 base pairs. In some embodiments, the deletion can include all or substantially all of the proviral sequence. In some embodiments the deletion can eradicate the provirus. The mutation can also comprise one or more insertions, that is, the addition of one or more nucleotide base pairs to the proviral sequence. The size of the inserted sequence also may vary, for example from about one base pair to about 300 nucleotide base pairs. The mutation can comprise one or more point mutations, that is, the replacement of a single nucleotide with another nucleotide. Useful point mutations are those that have functional consequences, for example, mutations that result in the conversion of an amino acid codon into a termination codon, or that result in the production of a nonfunctional protein.

In embodiments. the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

Three types (I-III) of CRISPR systems have been identified. CRISPR clusters contain spacers, the sequences complementary to antecedent mobile elements. CRISPR clusters are transcribed and processed into mature CRISPR RNA (crRNA). In embodiments, the CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

The wild type Streptococcus pyogenes Cas9 sequence can be modified. The nucleic acid sequence can be codon optimized for efficient expression in mammalian cells, i.e., “humanized.” sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the invention also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9(K855A), SpCas9(K810A/K1003A/R1060A) (a.k.a. eSpCas9 1.0), and SpCas9(K848A/K1003A/R1060A) (a.k.a. eSPCas9 1.1) are employed in the compositions. The invention is by no means limited to these variants, and also encompasses all Cas9 variants (Slaymaker, I. M. et al. (2015)).

The present invention also includes another type of enhanced specificity Cas9 variant, “high fidelity” spCas9 variants (HF-Cas9) (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

Guide Nucleic Acid Sequences:

Guide RNA sequences according to the present invention can be sense or anti-sense sequences. The specific sequence of the gRNA may vary, but, regardless of the sequence, useful guide RNA sequences will be those that minimize off-target effects while achieving high efficiency and complete ablation of the virus. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologues may have different PAM specificities. For example, Cas9 from S. thermophilus requires 5′-NNAGAA for CRISPR 1 and 5′-NGGNG for CRISPR3 and Neiseria meningitidis requires 5′-NNNNGATT. The specific sequence of the guide RNA may vary, but, regardless of the sequence, useful guide RNA sequences will be those that minimize off-target effects while achieving high efficiency and complete ablation of the Flavivirus, for example, the Zika virus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. In certain embodiments, the composition comprises multiple different gRNA molecules, each targeted to a different target sequence. In certain embodiments, this multiplexed strategy provides for increased efficacy. These multiplex gRNAs can be expressed separately in different vectors or expressed in one single vector.

The compositions and methods of the present invention may include a sequence encoding a guide RNA that is complementary to a target sequence in a Flavivirus. Flaviviruses included within the scope of this invention are discussed generally in Fields Virology, Editors: Fields, N., Knipe, D. M. and Howley, P. M.; Lippincott-Raven Publishers, Philadelphia, Pa.; Chapter 31 (1996). Specific flaviviruses include, without limitation: Absettarov; Alfuy; Apoi; Aroa; Bagaza; Banzi; Bououi; Bussuquara; Cacipacore; Carey Island; Dakar bat; Dengue viruses 1, 2, 3 and 4; Edge Hill; Entebbe bat; Gadgets Gully; Hanzalova; Hypr; Ilheus; Israel Turkey meningoencephalitis; Japanese encephalitis; Jugra; Jutiapa; Kadam; Karshi; Kedougou; Kokoera; Koutango; Kumlinge; Kunjin; Kyasanur Forest virus; Langat; Louping ill; Meaban; Modoc; Montana myotis leukoencephalitis; Murray valley encephalitis; Naranj al; Negishi; Ntaya; Omsk hemorrhagic fever; Phnom-Penh bat; Powassan; Rio Bravo; Rocio; Royal Farm; Russian spring-summer encephalitis; Saboya; St. Louis encephalitis; Sal Vieja; San Perlita; Saumarez Reef; Sepik; Sokuluk; Spondweni; Stratford; Temusu; Tyuleniy; Uganda S, Usutu, Wesselsbron; West Nile; Yaounde; Yellow fever; and Zika.

In certain embodiments, the Flavirus comprises: Dengue Fever Virus, West Nile Fever Virus, Yellow Fever Virus, St. Louis Encephalitis Virus, Japanese Encephalitis Virus, Murray Valley Encephalitis Virus, Tick-borne Encephalitis Virus, Kunjin Encephalitis Virus, Rocio Encephalitis Virus, Russian Spring Summer Encephalitis Virus, Negishi Virus, Kyasanur Forest Virus, Omsk Hemorrhagic Fever Virus, Powassan Virus, Louping III Virus, Rio Bravo Virus, Tyuleniy Virus, Ntaya Virus, Modoc Virus, Alkhurma Hemorrhagic Fever Virus, Zika virus.

In one embodiment, the Flavivirus is Zika virus.

In certain embodiments, a composition for eradicating a flavivirus in vitro or in vivo, comprises a therapeutically effective amount of: an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Flavivirus genome; an anti-viral agent or combinations thereof. In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

In certain embodiments, the anti-viral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, ribozymes, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

In certain embodiments, the antiviral agent comprises natural or recombinant interferon-alpha (IFNα), interferon-beta (IFNβ), interferon-gamma (IFNγ), interferon tau (IFNτ), interferon omega (IFNω), or combinations thereof. In some embodiments, the interferon is IFNγ. Any of these interferons can be stabilized or otherwise modified to improve the tolerance and biological stability or other biological properties. One common modification is pegylation (modification with polyethylene glycol).

In certain embodiments, the isolated nucleic acid sequence further comprises a short proto-spacer adjacent motif (PAM)-presenting DNA oligonucleotide sequence (PAMmer). As used herein the “PAMmer” is an oligonucleotide comprising a PAM and additional Flavivirus sequences, e.g. Zika sequences, downstream of the target Flavivirus sequences, e.g. Zika sequences, of the gRNA.

In another embodiment, a composition comprises an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Flavivirus genome; an anti-viral agent; an anti-pyretic agent, anti-inflammatory agent, chemotherapeutic agent, or combinations thereof.

In another embodiment, a target nucleic acid sequence comprises one or more nucleic acid sequences in coding and non-coding nucleic acid sequences of the Flavivirus genome. The target nucleic acid sequence can be located within a sequence encoding structural proteins, non-structural proteins or combinations thereof. The sequences encoding structural proteins comprise nucleic acid sequences encoding a capsid protein (C), precursor viral membrane protein (prM), viral membrane protein (M), envelop protein (E) or combinations thereof. The sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In certain embodiments, a gRNA sequence has at least a 75% sequence identity to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

Non-limiting examples of gRNA nucleic acid sequences are as follows:

(SEQ ID NO: 1)
5′-GTGAGTCAGACTGCGACAGTTCGAGT-3′
(SEQ ID NO: 2)
3′-CACTCAGTCTGACGCTGACAAGCTCA-5′
(SEQ ID NO: 3)
5′-TTAATTTGGATTTGGAAACGAGAGT-3′
(SEQ ID NO: 4)
3′-AATTAAACCTAAACCTTTGCTCTCA-5
(SEQ ID NO: 5)
5′-ACCCCACGCGCTTGGAAGCGCAGGAT-3′
(SEQ ID NO: 6)
3′-TGGGGTGCGCGAACCTTCGCGTCCTA-5′
(SEQ ID NO: 7)
5′-GCCTGAACTGGAGACTAGCTGTGAAT-3′
(SEQ ID NO: 8)
3′-CGGACTTGACCTCTGATCGACACTTA-5′
(SEQ ID NO: 9)
5′-ATGCTGTTTTGCGTTTTCCGGGGGGT-3′
(SEQ ID NO: 10)
3′-TACGACAAAACGCAAAAGGCCCCCCA-5′
(SEQ ID NO: 11)
5′-CCGATCCTAGACAAATGTGGAAGAGT-3′
(SEQ ID NO: 12)
3′-GGCTAGGATCTGTTTACACCTTCTCA-5′
(SEQ ID NO: 13)
5′-TCACGCTTACTACAACCCATCAGAGT-3′
(SEQ ID NO: 14)
3′-AGTGCGAATGATGTTGGGTAGTCTCA-5′
(SEQ ID NO: 15)
5′-GCATTAGTAAGTTTGATCTGGAGAAT-3′
(SEQ ID NO: 16)
3′-CGTAATCATTCAAACTAGACCTCTTA-5′
(SEQ ID NO: 17)
5′-ACAGGAGTGGAAACCCTCGACTGGAT-3′
(SEQ ID NO: 18)
3′-TGTCCTCACCTTTGGGAGCTGACCTA-5′

Table 1 provides non-limiting examples of RNA-guided Cas9 which cleaves ssRNA targets in the presence of a short PAM-presenting DNA oligonucleotide (PAMmer).

TABLE 1

| Targeted | | |
|---|---|---|
| region | motif | corresponding PAMmer sequence |
| 5′-UTR | 1 | 5′-TCGAGTCTGAAGCGAGAGCT-3′ |
| | | (SEQ ID NO: 19) |
| 5′-UTR | 2 | 5′-GAGAGTTTCTGGTCATGAAA-3′ |
| | | (SEQ ID NO: 20) |
| 3′-UTR | 1 | 5′-CAGGATGGGAAAAGAAGGTG-3′ |
| | | (SEQ ID NO: 21) |
| 3′-UTR | 2 | 5′-GTGAATCTCCAGCAGAGGGA-3′ |
| | | (SEQ ID NO: 22) |
| 3′-UTR | 3 | 5′-GGGGGGTCTCCTCTAACCAC-3′ |
| | | (SEQ ID NO: 23) |
| NS3 | 1 | 5′-AAGAGTGATAGGACTCTATG-3′ |
| | | (SEQ ID NO: 24) |
| NS3 | 2 | 5′-CAGAGTCCCTAATTACAATC-3′ |
| | | (SEQ ID NO: 25) |
| NS5 | 1 | 5′-GAGAATGAAGCTCTGATTAC-3′ |
| | | (SEQ ID NO: 26) |
| NS5 | 2 | 5′-CTGGATGGAGCAATTGGGAA-3′ |
| | | (SEQ ID NO: 27) |

In other embodiments, the gRNA sequences have at least a 75% sequence identity to sequences comprising: SEQ ID NOS: 1-18, or combinations thereof. In other embodiments, the gRNA sequences comprise: SEQ ID NOS: 1-18, or combinations thereof.

In other embodiments, the isolated nucleic acid sequences further comprise a short proto-spacer adjacent motif (PAM)-presenting DNA oligonucleotide sequence (PAMmer) wherein the PAMmer oligonucleotides comprise a PAM and additional Zika sequences downstream of the target Zika sequences of the gRNA. In embodiments, the Zika sequences comprise sequences within coding and non-coding nucleic acid sequences. In other embodiments the nucleic acid sequences are located within nucleic acid sequences encoding structural and non-structural proteins. In certain embodiments, the short PAM-presenting DNA oligonucleotide sequences (PAMmer) have at least a 75% sequence identity to at least one nucleic sequence comprising: SEQ ID NOS: 19-27, or combinations thereof. In other embodiments, the PAMmer sequences comprise at least one of SEQ ID NOS: 19-27, or combinations thereof.

In certain embodiments, an isolated nucleic acid sequence comprises a nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome. In other embodiments, the isolated nucleic acid sequence further comprises one or more PAMmer nucleic acid sequences.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the gRNA sequences or in a separate vector.

Modified or Mutated Nucleic Acid Sequences:

In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, tracrRNA etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O—P—O—CH). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Delivery Vehicles

Delivery vehicles as used herein, include any types of molecules for delivery of the compositions embodied herein, both for in vitro or in vivo delivery. Examples, include, without limitation: expression vectors, nanoparticles, colloidal compositions, lipids, liposomes, nanosomes, carbohydrates, organic or inorganic compositions and the like.

In some embodiments, a delivery vehicle is an expression vector, wherein the expression vector comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the isolated nucleic acid sequences for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). AAV include serotypes 1 through 9. An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and a guide RNA complementary to a target sequence of a Flavivirus, as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of, for example the Zika virus. The topical gel can be applied directly to the skin or mucous membranes of the male or female genital region prior to sexual activity. Alternatively, or in addition the topical gel can be applied to the surface or contained within a male or female condom or diaphragm.

In some embodiments, the compositions can be formulated as a nanoparticle encapsulating the compositions embodied herein.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more Cas/gRNA vectors. In some embodiments, the methods of the invention can be applied ex vivo. That is, a subject's cells can be removed from the body and treated with the compositions in culture to excise, for example, Zika virus sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The isolated nucleic acids can be easily delivered to a subject by methods known in the art, for example, methods which deliver siRNA. In some aspects, the Cas may be a fragment wherein the active domains of the Cas molecule are included, thereby cutting down on the size of the molecule. Thus, the, Cas9/gRNA molecules can be used clinically, similar to the approaches taken by current gene therapy. In particular, a Cas9/multiplex gRNA stable expression stem cell or iPS cells for cell transplantation therapy as well as vaccination can be developed for use in subjects.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Methods of Treatment

In certain embodiments, a method of eradicating a Flavivirus genome in a cell or a subject, comprises contacting the cell or administering to the subject, a pharmaceutical composition comprising a therapeutically effective amount of a gene editing agent and at least one guide RNA (gRNA), the gRNA being complementary to a target nucleic acid sequence in a Flavivirus genome.

In certain embodiments, a method of eradicating a Flavivirus genome in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

In other embodiments, a method of inhibiting replication of a Flavivirus in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

In other embodiments, a method of inhibiting replication of a Flavivirus in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Flavivirus genome, an anti-viral agent, or combinations thereof. In certain embodiments, a method of eradicating a Flavivirus genome in a cell or a subject, comprises contacting the cell or administering to the subject, a pharmaceutical composition comprising a therapeutically effective amount of a gene editing agent; at least one guide RNA (gRNA), the gRNA being complementary to a target nucleic acid sequence in a Flavivirus genome, an anti-viral agent, or combinations thereof. In addition, one or more therapeutic agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, antibiotics, or combinations thereof.

In certain embodiments, a method of eradicating a Flavivirus genome in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome an anti-viral agent, or combinations thereof. In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

The compositions of the present invention can be prepared in a variety of ways known to one of ordinary skill in the art. Regardless of their original source or the manner in which they are obtained, the compositions disclosed herein can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a Zika viral infection or at risk for contracting a Zika virus infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more an antiviral agent, or combinations thereof in pharmaceutically acceptable carriers. In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, antibiotics or combinations thereof.

In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

Kits

The compositions described herein can be packaged in suitable containers labeled, for example, for use as a therapy to treat a subject having a flavivirus infection, for example, a Zika virus infection or a subject at risk of contracting for example, a Zika virus infection. The containers can include a composition comprising a polypeptide or a nucleic acid sequence encoding a gene editing agent, e.g. an expression vector encoding a CRISPR-associated endonuclease, for example, a Cas9 endonuclease, a guide RNA complementary to a target sequence in a flavivirus virus and one or more of a suitable stabilizer, carrier molecule, flavoring, and/or the like, as appropriate for the intended use. In another embodiment, a first vector encodes for a CRISPR-associated endonuclease, a second vector encoding one or more gRNAs; or, separate vectors encoding one or more gRNAs. In other embodiments, the kit further comprises one or more anti-viral agents and/or therapeutic reagents that alleviate some of the symptoms or secondary bacterial infections that may be associated with a flavivirus infection. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding a CRISPR-associated endonuclease, for example, a Cas9 endonuclease, and a guide RNA complementary to a target sequence in a Zika virus, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The present invention is further illustrated by the following specific examples. The examples are provided for illustration only and are not to be construed as limiting the scope or content of the invention in any way.

### Example 1: Zika Virus Replication and Viral Propagation is Suppressed by the Combination of IFN-Gamma and CRISPR/Cas9 Mediated Gene Editing Strategy

Materials and Methods:

In order to investigate the possible impact of IFN-gamma and CRISPR/Cas9 mediated gene editing on Zika virus replication, primary human fetal astrocytes were cultured and plated in 6-well tissue culture dishes. When the cells reached a confluency of 80%, they were infected with Zika virus (ATCC® Number: VR-1843™, Strain: PRVABC59, Lot#: 64104231) at 0.2 MOI in serum free OPTI-MEM™, media for two hours. Infection mixtures were removed and fresh media with serum were added. At 24 hrs post-infections, cells were transfected with gRNAs and a plasmid encoding Cas9 endonuclease in the presence or absence of 20 ng/ml recombinant human IFN-gamma (EMD Millipore, IF002). IFN-gamma treatments were repeated at 2 dpi and 3 dpi in order to maintain IFN-gamma in culture media. At 4 dpi post-infections, culture media of cells were collected, centrifuged at 10,000 rpm for 10 minutes, and boiled at 95° C. for the inactivation of virus. Q-RT-PCR was performed (as described by Garcez P. P. et al., Science 10.1126/science.aaf6116 (2016)) to determine viral copy numbers in media along with samples from uninfected control cells.

Results:

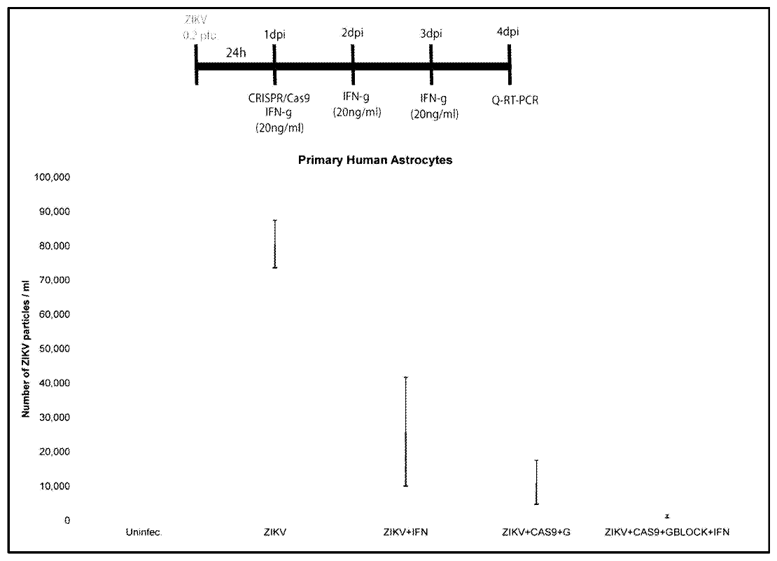

These results provide evidence that Zika virus can actively replicate and cause lytic infection in both astrocyte and microglia cells. Interestingly, astrocytes are more susceptible to Zika virus replication than microglial cells. In order to gain insight into the possible infection of human primary glial cells, human astrocytes and microglial cells were infected with Zika virus. The results evidence that Zika virus can actively replicate and cause lytic infection in both astrocyte and microglia cells. Interestingly, astrocytes are more susceptible to Zika virus replication than microglial cells. As shown in FIG. 1, uninfected PHFA cells were negative for Zika virus. On the other hand, astrocytes infected with Zika virus showed a robust replication of Zika virus as evidenced for the detection of viral particles in culture media. Interestingly, treatment of cells with IFN-gamma for the duration of infections resulted in a major and significant reduction in the numbers of viral particles in culture media suggesting anti-Zika virus activity of IFN-gamma. On the other hand, cells treated with CRISPR/Cas9 and gRNAs targeting Zika virus showed even greater reductions in viral copy numbers. Moreover, cells treated with both IFN-gamma and CRISPR-Cas9 constructs represented only trace numbers of Zika virus particles in the growth media providing evidence that combination therapies including IFN-gamma and CRISPR/Cas9 can block Zika virus replication and protect against new infections.

Discussion

These data indicate that both IFN-gamma and CRISPR/Cas9 can suppress Zika virus replication in astrocytes. IFN-gamma and CRISPR/Cas9 utilize different mechanisms to suppress the virus. These results provide evidence that IFN-gamma can target protein translation machinery and put a block on viral protein translation leading to reduced genomic replication and virion production. IFN-gamma shows no direct effect on viral genome or proteins already present in the infected cells. It will simply suppress the production and replication of new viral copies. On the other hand, CRISPR/Cas9 approach is designed to directly target viral genomes existing in infected cells. CRISPR/Cas9 will utilize specific gRNA sequences to target and cleave viral genome. The efficiency of CRISPR/Cas9 approach is dependent on the quantity of genomic copy numbers in the infected cells. By combining IFN-gamma with CRISPR/Cas9 approach, ensures keeping Zika viral copies low at constant levels with IFN-gamma then apply CRISPR/Cas9 approach for the complete elimination of virus from infected cells. The combination of these two approaches with distinct mechanisms provides superior benefits for the suppression and inhibition of Zika virus.

What is claimed:

1. A composition for eradicating a flavivirus in vitro or in vivo, the composition comprising:
an isolated nucleic acid sequence encoding a Clustered Regularly Interspaced Short Palindromic Repeat (CRISPR)-associated endonuclease;
at least one guide RNA (gRNA), the gRNA being complementary to a target nucleic acid sequence in a Flavivirus genome;
an antiviral agent, or
combinations thereof.

2. The composition of claim 1, wherein the Flavivirus comprises: dengue virus, tick-borne encephalitis virus, West Nile virus, yellow fever virus, Japanese encephalitis virus, Kyasanur Forest disease virus, Alkhurma hemorrhagic fever virus, Omsk hemorrhagic fever virus, or Zika virus.

3. The composition of claim 1, wherein the Flavivirus is Zika virus.

4. The composition of claim 1, wherein the antiviral agent comprises: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, ribozymes, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

5. The composition of claim 4, wherein the antiviral agent comprises interferon-alpha (IFNα), interferon-beta (IFNβ), interferon-gamma (IFNγ), interferon tau (IFNτ), interferon omega (IFNω), or combinations thereof.

6. The composition of claim 5, wherein the anti-viral agent is interferon-gamma (IFNγ).

7. The composition of claim 1, wherein the target nucleic acid sequence comprises one or more nucleic acid sequences in coding and non-coding nucleic acid sequences of the Flavivirus genome.

8. The composition of claim 1, wherein the target nucleic acid sequence comprises one or more sequences within a sequence encoding structural proteins, non-structural proteins or combinations thereof.

9. The composition of claim 8, wherein the sequences encoding structural proteins comprise nucleic acid sequences encoding a capsid protein (C), precursor viral membrane protein (prM), viral membrane protein (M), envelop protein (E) or combinations thereof.

10. The composition of claim 9, wherein the sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

11. The composition of claim 1, wherein the gRNA sequence has at least a 75% sequence identity to one or more sequences complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or any combination thereof.

12. The composition of claim 11, wherein a gRNA has at least a 75% sequence identity to any one or more of SEQ ID NOS: 1-27.

13. The composition of claim 11, wherein a gRNA comprises any one or more of SEQ ID NOS: 1-27.

14. The composition of claim 1, further comprising a short proto-spacer adjacent motif (PAM)-presenting DNA oligonucleotide sequence (PAMmer) wherein the PAMmer comprises a PAM and additional Flavivirus nucleic acid sequences downstream of target Flavivirus nucleic acid sequences of the gRNA.

15. The composition of claim 1, wherein the guide RNA sequences are in single or multiplex configurations.

16. The composition of claim 1, wherein the guide RNA sequences comprise chimeric regions, modified nucleic acid bases or combinations thereof.

17. The composition of claim 15, wherein the guide RNA sequences are encoded by the same vector encoding the CRISPR/Cas molecule or are encoded by separate vectors.

18. The composition of claim 1, further comprising an anti-pyretic agent, anti-inflammatory agent, chemotherapeutic agent, or combinations thereof.

19. A method of eradicating a Flavivirus genome in a cell or a subject, comprising contacting the cell or administering to the subject, a therapeutically effective amount of a pharmaceutical composition comprising:
an isolated nucleic acid sequence encoding a Clustered Regularly Interspaced Short Palindromic Repeat (CRISPR)-associated endonuclease;
at least one guide RNA (gRNA), the gRNA being complementary to a target nucleic acid sequence in a Flavivirus genome;
an antiviral agent, or
combinations thereof.

20. The method of claim 20, wherein the Flavivirus comprises: dengue virus, tick-borne encephalitis virus, West Nile virus, yellow fever virus, Japanese encephalitis virus, Kyasanur Forest disease virus, Alkhurma hemorrhagic fever virus, Omsk hemorrhagic fever virus, or Zika virus.

21. The method of claim 20, wherein the Flavivirus is Zika virus.

22. The method of claim 20, wherein the gene editing agent and the at least one guide RNA are encoded by the same vector or a different vector.

23. The method of claim 20, wherein the guide RNA sequences are in single or multiplex configurations.

24. The method of claim 20, wherein the antiviral agent comprises: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, ribozymes, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

25. The method of claim 24, wherein the antiviral agent comprises interferon-alpha (IFNα), interferon-beta (IFNβ), interferon-gamma (IFNγ), interferon tau (IFNτ), interferon omega (IFNω), analogs or combinations thereof.

26. The method of claim 25, wherein the anti-viral agent is interferon-gamma (IFNγ).

27. The method of claim 20, wherein the target nucleic acid sequence comprises one or more nucleic acid sequences in coding and non-coding nucleic acid sequences of the Flavivirus genome.

28. The method of claim 20, wherein the target nucleic acid sequence comprises one or more sequences within a sequence encoding structural proteins, non-structural proteins or combinations thereof.

29. The method of claim 28, wherein the sequences encoding structural proteins comprise nucleic acid sequences encoding a capsid protein (C), precursor viral membrane protein (prM), viral membrane protein (M), envelop protein (E) or combinations thereof.

30. The method of claim 28, wherein the sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

31. The method of claim 29, wherein the at least one gRNA sequence has at least a 75% sequence identity to at least one sequence, the sequence being complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

32. The method of claim 20, wherein the guide RNA sequences comprise chimeric regions, modified nucleic acid bases or combinations thereof.

33. The method of claim 20, wherein a gRNA has at least a 75% sequence identity to any one or more of SEQ ID NOS: 1-27.

34. The method of claim 20, wherein a gRNA comprises any one or more of SEQ ID NOS: 1-27.

35. The method of claim 20, further comprising an anti-pyretic agent, anti-inflammatory agent, chemotherapeutic agent, or combinations thereof.

36. A method of inhibiting replication of a Flavivirus in a cell or a subject, comprising contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease;
at least one guide RNA (gRNA), the gRNA being complementary to a target nucleic acid sequence in a Flavivirus genome;
an antiviral agent,
an anti-pyretic agent, anti-inflammatory agent, chemotherapeutic agent, or
combinations thereof.

37. The method of claim 36, wherein the antiviral agent comprises: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

38. A composition for eradicating a flavivirus in vitro or in vivo, the composition comprising:
a gene editing agent;
at least one guide nucleic acid sequence (gNAS), the gNAS being complementary to a target nucleic acid sequence in a Flavivirus genome;
an antiviral agent, or
combinations thereof.

39. The composition of claim 38, wherein the gene-editing agent comprises: Argonaute family of endonucleases, clustered regularly interspaced short palindromic repeat (CRISPR) nucleases, zinc-finger nucleases (ZFNs), transcription activator-like effector nucleases (TALENs), meganucleases, endo- or exo-nucleases, or combinations thereof.

40. The composition of claim 38, wherein the gNAS comprises a ribonucleic acid (RNA) or deoxyribonucleic acid (DNA).

41. The composition of claim 38, wherein the gNAS comprises one or more modified nucleic acid bases or chimeric sequences.

42. The composition of claim 38, wherein the gene editing agent and the at least one gNAS is encoded by the same vector or separate vectors.

43. The composition of claim 38, wherein the guide NAS sequences are in single or multiplex configurations.

44. A method of treating a subject infected with a Zika virus, comprising:
administering to the subject, a pharmaceutical composition comprising a therapeutically effective amount of an isolated nucleic acid sequence encoding a Clustered Regularly Interspaced Short Palindromic Repeat (CRISPR)-associated endonuclease;
at least one guide RNA (gRNA), the gRNA being complementary to a target nucleic acid sequence in a Zika virus genome; and,
an antiviral agent.

45. The method of claim 44, wherein the antiviral agent comprises interferon-alpha (IFNα), interferon-beta (IFNβ), interferon-gamma (IFNγ), interferon tau (IFNτ), interferon omega (IFNω), analogs or combinations thereof.

46. The method of claim 44, wherein the anti-viral agent is interferon-gamma (IFNγ).

47. The method of claim 44, wherein the guide RNA sequences are in single or multiplex configurations.

48. A pharmaceutical composition comprising a therapeutically effective amount of an isolated nucleic acid sequence encoding a Clustered Regularly Interspaced Short Palindromic Repeat (CRISPR)-associated endonuclease; at least one guide RNA (gRNA), the gRNA being complementary to a target nucleic acid sequence in a Zika virus genome; and, an antiviral agent.

49. The pharmaceutical composition of claim 48, wherein the antiviral agent comprises interferon-alpha (IFNα), interferon-beta (IFNβ), interferon-gamma (IFNγ), interferon tau (IFNτ), interferon omega (IFNω), analogs or combinations thereof.

50. The pharmaceutical composition of claim 48, wherein the anti-viral agent is interferon-gamma (IFNγ).

51. The pharmaceutical composition of claim 48, wherein the guide RNA sequences are in single or multiplex configurations.

52. The pharmaceutical composition of claim 48, wherein the target nucleic acid sequence comprises one or more nucleic acid sequences in coding and non-coding nucleic acid sequences of the Zika virus genome.

53. The pharmaceutical composition of claim 52, wherein the target nucleic acid sequence comprises one or more sequences within a sequence encoding structural proteins, non-structural proteins or combinations thereof.

54. The pharmaceutical composition of claim 53, wherein the sequences encoding structural proteins comprise nucleic acid sequences encoding a capsid protein (C), precursor viral membrane protein (prM), viral membrane protein (M), envelop protein (E) or combinations thereof.

55. The pharmaceutical composition of claim 53, wherein the sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

56. The pharmaceutical composition of claim 53, wherein the at least one gRNA sequence has at least a 75% sequence identity to at least one sequence, the sequence being complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

57. The pharmaceutical composition of claim 48, wherein a gRNA comprises one or more modified nucleic acid bases or chimeric sequences.

58. The pharmaceutical composition of claim 48, wherein a gRNA has at least a 75% sequence identity to any one or more of SEQ ID NOS: 1-27.

59. The pharmaceutical composition of claim 48, wherein a gRNA comprises any one or more of SEQ ID NOS: 1-27.

60. The pharmaceutical composition of claim 48, further comprising an anti-pyretic agent, anti-inflammatory agent, chemotherapeutic agent, or combinations thereof.


SEQUENCE LISTING (27 sequences)

SEQ ID NO: 1 - DNA, 26 bp, Artificial Sequence
  Description of Artificial Sequence Synthetic oligonucleotide
gtgagtcagactgcgacagttcgagt

SEQ ID NO: 2 - DNA, 26 bp, Artificial Sequence
  Description of Artificial Sequence Synthetic oligonucleotide
actcgaacagtcgcagtctgactcac

SEQ ID NO: 3 - DNA, 25 bp, Artificial Sequence
  Description of Artificial Sequence Synthetic oligonucleotide
ttaatttggatttggaaacgagagt

SEQ ID NO: 4 - DNA, 25 bp, Artificial Sequence
  Description of Artificial Sequence Synthetic oligonucleotide
actctcgtttccaaatccaaattaa

SEQ ID NO: 5 - DNA, 26 bp, Artificial Sequence
  Description of Artificial Sequence Synthetic oligonucleotide
accccacgcgcttggaagcgcaggat

SEQ ID NO: 6 - DNA, 26 bp, Artificial Sequence
  Description of Artificial Sequence Synthetic oligonucleotide
atcctgcgcttccaagcgcgtggggt

SEQ ID NO: 7 - DNA, 26 bp, Artificial Sequence
  Description of Artificial Sequence Synthetic oligonucleotide
gcctgaactggagactagctgtgaat

SEQ ID NO: 8 - DNA, 26 bp, Artificial Sequence
  Description of Artificial Sequence Synthetic oligonucleotide
attcacagctagtctccagttcaggc

SEQ ID NO: 9 - DNA, 26 bp, Artificial Sequence
  Description of Artificial Sequence Synthetic oligonucleotide
atgctgttttgcgttttccggggggt

SEQ ID NO: 10 - DNA, 26 bp, Artificial Sequence
  Description of Artificial Sequence Synthetic oligonucleotide
accccccggaaaacgcaaaacagcat

SEQ ID NO: 11 - DNA, 26 bp, Artificial Sequence
  Description of Artificial Sequence Synthetic oligonucleotide
ccgatcctagacaaatgtggaagagt

SEQ ID NO: 12 - DNA, 26 bp, Artificial Sequence
  Description of Artificial Sequence Synthetic oligonucleotide
actcttccacatttgtctaggatcgg

SEQ ID NO: 13 - DNA, 26 bp, Artificial Sequence
  Description of Artificial Sequence Synthetic oligonucleotide
tcacgcttactacaacccatcagagt

SEQ ID NO: 14 - DNA, 26 bp, Artificial Sequence
  Description of Artificial Sequence Synthetic oligonucleotide
actctgatgggttgtagtaagcgtga

SEQ ID NO: 15 - DNA, 26 bp, Artificial Sequence
  Description of Artificial Sequence Synthetic oligonucleotide
gcattagtaagtttgatctggagaat

SEQ ID NO: 16 - DNA, 26 bp, Artificial Sequence
  Description of Artificial Sequence Synthetic oligonucleotide
attctccagatcaaacttactaatgc

SEQ ID NO: 17 - DNA, 26 bp, Artificial Sequence
  Description of Artificial Sequence Synthetic oligonucleotide
acaggagtggaaaccctcgactggat

SEQ ID NO: 18 - DNA, 26 bp, Artificial Sequence
  Description of Artificial Sequence Synthetic oligonucleotide
atccagtcgagggtttccactcctgt

SEQ ID NO: 19 - DNA, 20 bp, Artificial Sequence
  Description of Artificial Sequence Synthetic oligonucleotide
tcgagtctgaagcgagagct

SEQ ID NO: 20 - DNA, 20 bp, Artificial Sequence
  Description of Artificial Sequence Synthetic oligonucleotide
gagagtttctggtcatgaaa

SEQ ID NO: 21 - DNA, 20 bp, Artificial Sequence
  Description of Artificial Sequence Synthetic oligonucleotide
caggatgggaaaagaaggtg

SEQ ID NO: 22 - DNA, 20 bp, Artificial Sequence
  Description of Artificial Sequence Synthetic oligonucleotide
gtgaatctccagcagaggga

SEQ ID NO: 23 - DNA, 20 bp, Artificial Sequence
  Description of Artificial Sequence Synthetic oligonucleotide
ggggggtctcctctaaccac

SEQ ID NO: 24 - DNA, 20 bp, Artificial Sequence
  Description of Artificial Sequence Synthetic oligonucleotide
aagagtgataggactctatg

SEQ ID NO: 25 - DNA, 20 bp, Artificial Sequence
  Description of Artificial Sequence Synthetic oligonucleotide
cagagtccctaattacaatc

SEQ ID NO: 26 - DNA, 20 bp, Artificial Sequence
  Description of Artificial Sequence Synthetic oligonucleotide
gagaatgaagctctgattac

SEQ ID NO: 27 - DNA, 20 bp, Artificial Sequence
  Description of Artificial Sequence Synthetic oligonucleotide
ctggatggagcaattgggaa